(19) United States
(12) Patent Application Publication
(10) Pub. No.: US 2013/0109680 A1
(43) Pub. Date: May 2, 2013

(54) METHODS AND COMPOSITIONS FOR AMELIORATING THIAZIDE INDUCED HYPERLIPIDEMIA

(71) Applicants: Richard Johnson, Centennial, CO (US); Takahiko Nakagawa, Greenwood Village, CO (US); Sirirat Reungjui, Gainesville, FL (US)

(72) Inventors: Richard Johnson, Centennial, CO (US); Takahiko Nakagawa, Greenwood Village, CO (US); Sirirat Reungjui, Gainesville, FL (US)

(73) Assignee: University of Florida Research Foundation, Inc., Gainesville, FL (US)

(21) Appl. No.: 13/598,304

(22) Filed: Aug. 29, 2012

Related U.S. Application Data
(62) Division of application No. 12/531,120, filed on Jan. 27, 2010, now abandoned.
(60) Provisional application No. 60/912,293, filed on Apr. 17, 2007.

(51) Int. Cl. A61K 31/549; A61K 31/519

(52) U.S. Cl. CPC A61K 31/549; A61K 31/519
USPC 514/223.5

(57) ABSTRACT

Male Disclosed herein are compositions for ameliorating the lipid producing effects of thiazide therapy. Particularly exemplified herein are compositions containing a thiazide and allopurinol, or some other xanthine oxidase inhibitor.

### RELATED APPLICATIONS

This application is a continuation of U.S. application Ser. No. 12/531,120 which is a national stage application of PCT application No. PCT/US08/60550 filed Apr. 17, 2008. This application claims priority to U.S. Ser. No. 60/912,293 filed Apr. 17, 2007, which is incorporated herein in its entirety.

### BACKGROUND

The metabolic syndrome (MetSyn) consists of a constellation of abnormalities that confer higher risks of cardiovascular disease and type 2 diabetes which include abdominal obesity, hypertriglyceridemia, low HDL cholesterol, hypertension and hyperglycemia (1-4). Other features associated with the MetSyn include a proinflammatory state, prothrombotic state, endothelial dysfunction, hyperuricemia and insulin resistance. In particular, endothelial dysfunction, hyperuricemia, and insulin resistance have all been proposed to have an underlying etiologic role in the MetSyn (3,5-9).

Hydrochlorothiazide (HCTZ) and the other thiazide-like diuretics have been shown to confer a beneficial effect in hypertension by reducing morbidity and mortality, especially as it relates to the frequency of stroke and congestive heart failure (10-12). However, HCTZ may also have several negative side-effects of the MetSyn. In addition to causing of volume depletion and electrolyte imbalance, especially hypokalemia, hyponatremia and hypomagnesemia, HCTZ causes hyperuricemia, hyperlipidemia and impairment of glucose metabolism (13-17). In spite of these adverse effects, HCTZ is still widely administered and remains an important therapy for treatment of hypertension (3). Therefore, it is important to understand the precise mechanism by which HCTZ exacerbates metabolic syndrome, which might reveal augmentation therapies to address the adverse effects of thiazide therapy. Thiazide induced-hypokalemia may mediate insulin resistance (18-19). In addition, experimental hyperuricemia can also cause endothelial dysfunction (5-20), hypertension (21) and hyperinsulinemia (5,22).

### SUMMARY

The subject invention is based on the inventors' discovery that critical adverse side effects associated with thiazide therapy are addressed with augmentation therapy using a xanthine oxidase inhibitor. One adverse side effect of particular concern is the increase of lipids in the blood or hyperlipidemia. In one embodiment, the subject invention relates to a method of ameliorating thiazide induced hyperlipidemia by administering a therapeutically effective amount of thiazide in conjunction with therapeutically effective amount of allopurinol in patients susceptible to thiazide induced hyperlipidemia.

In another embodiment, the subject invention pertains to a composition comprising a thiazide and an xanthine oxidase inhibitor. In a specific embodiment, the invention relates to a composition consisting essentially of a thiazide and an xanthine oxidase inhibitor; such composition may also include a non-active additive.

A further embodiment of the subject invention pertains to a composition consisting essentially of a thiazide and allopurinol.

In yet a further embodiment, the invention pertains to a composition consisting essentially of hydrochlorothiazide, chlorothiazide or a combination of both and allopurinol.

### BRIEF DESCRIPTION OF THE DRAWINGS

FIG. 1: Systolic blood pressure obtained from tail cuff sphygmomanometer at week 4 and week 16. The rats receiving fructose diet had hypertension which hydrochlorothiazide reduced the systolic blood pressure. Potassium supplement further reduced blood pressure demonstrated at week 16 while the effect of allopurinol on blood pressure reduction was shown at both weeks 4 and 16. Data are expressed as means±SD. p values at least <0.05; *p vs normal diet, ^{&} p vs fructose diet, ^{$}p vs F+HCTZ, ^{#}p vs F+HCTZ+KCL

FIG. 2: Time courses of serum uric acid (A), serum glucose (B), serum cholesterol (C) and serum triglycerides (D). The rats receiving fructose diet revealed hyperuricemia and hypertriglyceridemia throughout the study and hypercholesterolemia was detected at week 14. Hydrochlorothiazide added on fructose diet caused more hyperuricemia and hyperglycemia shown at weeks 14 and 20. Serum cholesterol and triglyceride were numerical higher with hydrochlorothiazide usage but not reached statistical significantly. Potassium supplement had a tendency of serum glucose reduction effect at week 20 (p=0.06) while allopurinol treatment reduced serum uric acid and triglyceride at all time points and showed significant reduction of serum glucose at week 20. Data are expressed as means±SD. p values at least <0.05; * p vs normal diet, ^{&}p vs fructose diet, ^{$}p vs F+HCTZ, ^{#}p vs F+HCTZ+KCL

FIG. 3: Quantitative Insulin Sensitivity Check Index (QUICKI) at week 14. The rats on fructose diet and hydrochlorothiazide had significant lower insulin sensitivity compared with the normal diet and the fructose diet groups. Treatment with allopurinol improved the insulin sensitivity. Data are expressed as means±SD. p values at least <0.05; * p vs normal diet, ^{&}p vs fructose diet, ^{$}p vs F+HCTZ.

FIG. 4: The insulin tolerance test at week 18. The fructose diet and fructose diet plus hydrochlorothiazide groups demonstrated no change of blood glucose to intraperitoneal insulin injection. Potassium supplement and allopurinol improved the insulin response similar with the normal diet group. Data are expressed as means±SD. p values at least <0.05; * p vs normal diet, ^{&} p vs fructose diet, ^{$}p vs F+HCTZ.

FIG. 5: Urine nitrate and nitrite the products of nitric oxide reaction. The rats receiving fructose diet had lower urine nitrate and nitrite than the normal diet group. Hydrochlorothiazide usage revealed more reduction of urine nitrate and nitrite. Potassium supplement and allopurinol increased the level of urine nitrate and nitrite compared with the fructose diet plus hydrochlorothiazide group. Data are expressed as means±SD. p values at least <0.05; *p vs normal diet, ^{&}p vs fructose diet, ^{$}p vs F+HCTZ

### DETAILED DESCRIPTION

The subject invention pertains to a conjunctive therapy for hypertension that ameliorates the lipid producing effects of thiazides. In one embodiment, the conjunctive therapy comprises the administering of a therapeutically effective amount of thiazide and co-administering a therapeutically effective amount of xanthine oxidase inhibitor. In a particular embodiment, the conjunctive therapy comprises administration of a composition comprising thiazide and allopurinol, or pharmaceutically acceptable salts thereof, as the primary active components. Thiazides are known to raise certain lipids and the inventors have discovered that xanthine oxidase inhibitors, such as allopurinol, counteract the lipid raising effects of thiazides.

In a further embodiment, a reduced-lipid producing thiazide containing pharmaceutical composition can be formulated in accordance with an ordinary method. Such a formulation can be produced usually by mixing/kneading active components, thiazide and xanthine oxidase inhibitor, with non-active additives such as an excipient, diluent and carrier. In this specification, a parenteral administration means to include subcutaneous injection, intravenous injection, intramuscular injection, intraperitoneal injection or dripping infusion and the like. A formulation for injection such as aseptic aqueous suspension or oily suspension for injection can be produced using a suitable dispersing agent or wetting agent and a suspending agent by a method known in the art. Such an aseptic formulation for injection may be an aseptic injectable solution or suspension in a diluent or solvent which can be non-toxic and administered parenterally, including an aqueous solution. An acceptable vehicle or solvent which can be employed may, for example, be water, Ringer's solution, isotonic saline and the like. An aseptic non-volatile oil can also be used usually as a solvent or suspending medium. For such purpose, any non-volatile oil or fatty acid can be employed, including naturally occurring or synthetic or semi-synthetic fatty oil or fatty acid, as well as naturally occurring or synthetic or semi-synthetic mono- or di- or tri-glycerides.

A suitable base (e.g. polymer of butyric acid, polymer of glycolic acid, copolymer of butyric acid and glycolic acid, mixture of a polymer of butyric acid and a polymer of glycolic acid, polyglycerol fatty acid ester and the like) may be combined to form a sustained release formulation.

In another embodiment, a solid dosage form for oral administration may, for example, be a powder, granule, tablet, pill, capsule and the like, as described above. The formulation of such a dosage form can be produced by mixing and/or kneading active compounds, thiazide and xanthine oxidase inhibitor, with at least one of the non-active additives, such as sucrose, milk sugar (lactose), cellulosic saccharide, mannitol (D-mannitol), maltitol, dextran, starches (e.g., corn starch), microcrystalline cellulose, agar, alginates, chitins, chitosans, pectins, tragacanth gums, gum arabic, gelatins, collagens, casein, albumin, synthetic or semi-synthetic polymers or glycerides. Such a dosage form can further contain additives as usual, including inert diluents, lubricants such as magnesium stearate, preservatives such as parabens and sorbic acid, antioxidants such as ascorbic acid, α-tocopherol and cysteine, disintegrants (e.g., croscarmellose sodium), binder (e.g., hydroxypropyl cellulose), thickening agents, buffering agents, sweeteners, flavoring agents, perfumes and the like. A tablet and pill may further be enteric-coated. An oral liquid formulation may, for example, be a pharmaceutically acceptable emulsion, syrup, elixir, suspension, solution and the like, which may contain a pharmaceutically customary inert diluent such as water and if desired, additives. Such an oral liquid formulation can be produced by mixing an active ingredient, inert diluent and other additives if necessary in accordance with a customary method. An oral formulation usually contain about 0.01 to 99% by weight, preferably about 0.1 to 90% by weight, usually about 0.5 to 50% by weight of an inventive active compound, although the amount may vary depending on the dosage form.

In an alternative embodiment, a suppository for rectal administration can be produced by mixing active components with a suitable non-irritating excipient which is solid at ambient temperature but becomes liquid at the temperature in an intestinal tract to melt in rectum whereby releasing the active ingredient, such as cocoa butter and polyethylene glycols.

The dose in a certain patient is determined considering the age, body weight, general condition, sex, diet, administration time, administration mode, excretion rate, drug combination, degree of the disease treated currently as well as other factors.

A reduced-lipid producing thiazide containing composition of the present invention has a low toxicity and can be used safely, and its daily dose varies depending on the condition and body weight of the patient, the type of the compound and the administration route and, for example, when used as a prophylactic and therapeutic against thiazide induced hyperlipidemia, it may be about 1 to 500 mg, preferably about 10 to 200 mg as an active ingredient [I] in an oral formulation, and about 0.1 to 100 mg, preferably about 1 to 50 mg, usually about 1 to 20 mg as an active ingredient [I] in a parenteral formulation for an adult (60 kg), a dose within which exhibited no toxicity.

Examples of xanthine oxidase inhibitors suitable for use in the reduced-lipid producing thiazide containing composition include, but are not limited to Allopurinol, hydroxyakalone, TEI-6720, carprofen, febuxostat, RDEA-806 (Andrea Bio), banaba, (whole plants, powder extracts, or isolated compounds) oral uricase, and y-700. U.S. Pat. No. 5,614,520 and U.S. Patent Pub. No. 2005/0090472 are cited for a non-limiting list of other examples.

Examples of thiazide diuretics include, but are not limited to, chlorothiazide, benzylhydro-chlorothiazide, cyclopenthiazide, ethiazide, hydrochlorothiazide, hydroflumethiazide, methyclothiazide, penfluthiazide, polythiazide, trichloromethiazide and the like.

In another embodiment, the subject invention pertains to a method of treating hypertension comprising administering a therapeutically effective amount of a thiazide, or pharmaceutically acceptable salt thereof and coadministering a therapeutically effective amount of allopurinol, or a pharmaceutically acceptable salt thereof, wherein said therapeutically effective amount of allopurinol, or pharmaceutically acceptable salt thereof, comprises an amount sufficient to reduce the lipid raising effects of said administering step.

The term “coadministering” or “concurrent administration”, when used, for example with respect to administration of a xanthine oxidase inhibitor along with administration of a thiazide refers to administration of the thiazide and the xanthine oxidase inhibitor such that both can simultaneously achieve a physiological effect. The two agents, however, need not be administered together. In certain embodiments, administration of one agent can precede administration of the other, however, such coadministering typically results in both agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

The administration mode of the conjunctive formulation of the present invention is not particularly limited, provided that the compound of the present invention and the conjunctive drug are combined upon administration. Such an administration mode may, for example, be (1) an administration of a single formulation obtained by formulating a thiazide and xanthine oxidase inhibitor simultaneously, (2) a simultaneous administration via an identical route of two formulations obtained by formulating a thiazide and a xanthine oxidase inhibitor separately, (3) a sequential and intermittent administration via an identical route of two formulations obtained by formulating a thiazide and a xanthine oxidase inhibitor separately, (4) a simultaneous administration via different routes of two formulations obtained by formulating a thiazide and a xanthine oxidase inhibitor separately, (5) a sequential and intermittent administration via different routes of two formulations obtained by formulating thiazide and a xanthine oxidase inhibitor separately (for example, thiazide or its pharmaceutical composition followed by xanthine oxidase inhibitor or its pharmaceutical composition, or inverse order) and the like.

A reduced-lipid producing thiazide containing composition of the present invention has a low toxicity, and thus a thiazide and xanthine oxidase inhibitor (e.g. allopurinol) are mixed with a pharmacologically acceptable carrier in accordance with a method known per se to form a pharmaceutical composition, for example, a tablet (including sugar-coated and film-coated tablets), powder, granule, capsule (including soft capsule), solution, injection formulation, suppository, sustained release formulation and the like, which can safely be given orally or parenterally (e.g., topically, rectally, intravenously). An injection formulation may be given intravenously, intramuscularly, subcutaneously, into an organ, or directly into a lesion.

A pharmacologically acceptable carrier which may be employed for producing a conjunctive formulation of the present invention may, for example, be various organic and inorganic carrier materials employed customarily as pharmaceutical materials such as excipients, lubricants, binders and disintegrants in a solid formulation, solvents, dissolution aids, suspending agents, isotonicity imparting agents, bufferring agents and analgesic agents in a liquid formulation. Furthermore, other additives such as ordinary preservatives, antioxidants, colorants, sweeteners, adsorbents, wetting agents may also be added in suitable amounts.

An excipient may, for example, be lactose, sugar, D-mannitol, starch, corn starch, crystalline cellulose, light silicate anhydride and the like.

A lubricant may, for example, be magnesium stearate, calcium stearate, talc, colloidal silica and the like.

A binder may, for example, be crystalline cellulose, sugar, D-mannitol, dextrin, hydroxypropyl cellulose, hydroxypropylmethyl cellulose, polyvinyl pyrrolidone, starch, sucrose, gelatin, methyl cellulose, sodium carboxymethyl cellulose and the like.

A disintegrant may, for example, be starch, carboxymethyl cellulose, calcium carboxymethyl cellulose, sodium carboxymethyl starch, L-hydroxypropyl cellulose and the like.

A solvent may, for example, be water for injection, alcohol, propylene glycol, macrogol, sesame oil, corn oil, olive oil and the like.

A dissolution aid may, for example, be polyethylene glycol, propylene glycol, D-mannitol, benzyl benzoate, ethanol, trisaminomethane, cholesterol, triethanolamine, sodium carbonate, sodium citrate and the like.

A suspending agent may, for example, be a surfactant such as stearyl triethanolamine, sodium lauryl sulfate, laurylaminopropionic acid, lecithin, benzalkonium chloride, benzethonium chloride, glycerin monostearate and the like; hydrophilic polymer such as polyvinyl alcohol, polyvinyl pyrrolidone, sodium carboxymethyl cellulose, methyl cellulose, hydroxymethyl cellulose, hydroxyethyl cellulose, hydroxypropyl cellulose and the like.

An isotonicity imparting agent may, for example, be glucose, D-sorbitol, sodium chloride, glycerin, D-mannitol and the like.

A buffering agent may, for example, be a buffer solution of a phosphate, acetate, carbonate, citrate and the like.

An analgesic may, for example, be benzyl alcohol.

A preservative may, for example, be a p-oxybenzoate, chlorobutanol, benzyl alcohol, phenethyl alcohol, dehydroacetic acid, sorbic acid and the like.

An antioxidant may, for example, be a sulfite, ascorbic acid, α-tocopherol, EGCG and the like.

The ratio between a thiazide and a xanthine oxidase inhibitor in a conjunctive formulation of the present invention may be selected appropriately on the basis of the target and route. For example, the amount of a thiazide is usually about 0.01 to 100% by weight, preferably about 0.1 to about 50% by weight, more preferably about 0.5 to about 20% by weight based on the entire formulation, although it may vary depending on the dosage form. The amount of a xanthine oxidase inhibitor is usually about 0.01 to 100% by weight, preferably about 0.1 to about 50% by weight, more preferably about 0.5 to about 20% by weight based on the entire formulation, although it may vary depending on the dosage form.

The amount of an additive such as a carrier contained in a conjunctive formulation of the present invention is usually about 1 to about 99.99% by weight, preferably about 10 to about 90% by weight based on the entire formulation, although it may vary depending on the dosage form.

Similar amounts may be employed also when a compound of the present invention and a conjunctive drug are formulated separately.

Such a formulation can be produced by a method known per se which is employed usually in a pharmaceutical process.

For example, a compound of the present invention and a conjunctive drug can be formulated with a dispersant (e.g., Tween 80 (ATLAS POWDER, USA), HCO60 (NIKKO CHEMICALS), polyethylene glycol, carboxymethyl cellulose, sodium alginate, hydroxypropylmethyl cellulose, dextrin), a stabilizer (e.g., ascorbic acid, sodium pyrosulfite), a surfactant (e.g., polysorbate 80, macrogol), a solubilizing agent (e.g., glycerin, ethanol), a buffering agent (phosphoric acid and its alkali metal salt, citric acid and its alkali metal salt and the like), an isotonizing agent (e.g., sodium chloride, potassium chloride, mannitol, sorbitol, glucose), a pH modifier (e.g., hydrochloric acid, sodium hydroxide), a preservative (e.g., ethyl p-oxybenzoate, benzoic acid, methylparabene, propylparabene, benzyl alcohol), a solubilizer (e.g., concentrated glycerin, meglumine), a solubilizing aid (e.g., propylene glycol, sugar), an analgesic (e.g., glucose, benzyl alcohol) into an aqueous formulation for injection, or dissolved, suspended or emulsified in a vegetable oil such as olive oil, sesame oil, cottonseed oil and corn oil and in a solubilizing aid such as propylene glycol to form an oily formulation, whereby producing an injection formulation.

In order to obtain an oral dosage form, a method known per se is employed to compact an inventive compound or a conjunctive drug for example with an excipient (e.g., lactose, sugar, starch), a disintegrant (e.g., starch, calcium carbonate), a binder (e.g., starch, gum Arabic, carboxymethyl cellulose, polyvinyl pyrrolidone, hydroxypropyl cellulose) or a glidant (e.g., talc, magnesium stearate, polyethylene glycol 6000) into a desired shape, which is then, if necessary, coated for the purpose of a taste masking, an enteric property or a sustained release performance by means of a coating method known per se, whereby obtaining an oral dosage form. Such a coating may, for example, be hydroxypropylmethyl cellulose, ethyl cellulose, hydroxymethyl cellulose, hydroxypropyl cellulose, polyoxyehtylene glycol, Tween 80, Pluronic F68, cellulose acetate phthalate, hydroxypropylmethyl cellulose phthalate, hydroxymethyl cellulose acetate succinate, Eudragit (Rohm, German, methacrylic/acrylic acid copolymer) and a colorant (e.g., iron oxide red, titanium dioxide). An oral dosage form may be an instantaneous release formulation or a sustained release formulation.

While the dose of an inventive conjunctive formulation may vary depending on the type of the inventive compound, the subject's age, body weight, condition, and the dosage form as well as administration mode and duration, for example, the daily dose in a patient having hyperlipidemia (adult, body weight: about 60 kg) is about 0.01 to about 1000 mg/kg, preferably about 0.01 to about 100 mg/kg, more preferably about 0.1 to about 100 mg/kg, particularly about 0.1 to about 50 mg/kg, especially about 1.5 to about 30 mg/kg as an inventive compound, which is given intravenously at once or in several portions. It is a matter of course that the dose may vary depending on various factors as described above, and a less amount may sometimes be sufficient and an excessive amount should sometimes be required.

A conjunctive drug (i.e. xanthine oxidase inhibitor) may be employed in any amount within the range causing no problematic side effects. The daily dose of a conjunctive drug is not limited particularly and may vary depending on the severity of the disease, the subject's age, sex, body weight and susceptibility as well as time and interval of the administration and the characteristics, preparation, type and active ingredient of the pharmaceutical formulation, and the daily oral dose per kg body weight in a mammal is about 0.001 to 2000 mg, preferably about 0.01 to 500 mg, more preferably about 0.1 to about 100 mg as medicaments, which is given usually in 1 to 4 portions.

When an inventive conjunctive formulation is administered, it may be administered at the same time, but it is also possible that a conjunctive drug is first administered and then an inventive compound is administered, or that the inventive compound is first administered and then the conjunctive drug is administered. When such an intermittent administration is employed, the time interval may vary depending on the active ingredient administered, the dosage form and the administration mode, and for example, when the conjunctive drug is first administered, the inventive compound may be administered within 1 minute to 3 days, preferably 10 minutes to 1 day, more preferably 15 minutes to 1 hour after the administration of the conjunctive drug. When the inventive compound is first administered, for example, then the conjunctive drug may be administered within 1 minute to 1 day, preferably 10 minutes to 6 hours, more preferably 15 minutes to 1 hour after the administration of the inventive compound.

In a preferred administration mode, for example, about 0.001 to 200 mg/kg of a conjunctive drug formulated as an oral formulation is given orally as a daily dose, and, after about 15 minutes, about 0.005 to 100 mg/kg of an inventive compound formulated as an oral formulation is given orally as a daily dose.

Additional pharmacologically active agents may be delivered along with the primary active agents, thiazide and xanthine oxidase inhibitors. In one embodiment, such agents include, but are not limited to beta blockers, statins, and aspirin. Suitable statins are well known to those of skill in the art. Such statins include, but are not limited to atorvastatin (LIPITOR®, Pfizer), simvastatin (ZOCOR®, Merck®, pravastatin (PRAVACHOL®, Bristol-Myers Squibb), fluvastatin (LESCOL®, Novartis), lovastatin (MEVACOR®, Merck), rosuvastatin (Crestor®, Astra Zeneca), and Pitavastatin (Sankyo), and the like. Suitable beta blockers include, but are not limited to cardioselective (selective beta 1 blockers), e.g., acebutolol, atenolol, betaxolol, bisoprolol, metoprolol, and the like. Suitable non-selective blockers (block beta 1 and beta 2 equally) include, but are not limited to carteolol, nadolol, penbutolol, pindolol, propranolol, timolol, labetalol,and the like.

The invention is further detailed in the following Examples, Formulation Examples and Experiments, which are not intended to restrict the invention.

A value indicated for a solvent mixture is a volume ratio of each solvent, unless otherwise specified. A % is a % by weight, unless otherwise specified. A ratio of the elution solvent in a chromatography on a silica gel is a volume ratio, unless otherwise specified. Room temperature (ambient temperature) employed here usually means a temperature from about 20 to about 30° C.

### EXAMPLE

### Reduction of Thiazide Induced Hyperlipidemia

Materials and Methods

Experimental Protocol

All animal studies were approved by the University of Florida Institutional Animal Use and Care Committee (IACUC).

Male Sprague Dawley rats (150-200 g, Charles River Inc., Wilmington, Mass.) were placed on a standard natural ingredient diet (catalog #7912, Harlan, Wis.) for a five day run-in period with collection of basal blood and urine. Rats were then divided into 5 groups with similar body weight and baseline blood chemistry (n=8 each group).

Group 1 (N): normal standard diet

Group 2 (F): 60% fructose diet (TD.89247, Harlan, Wis.)

Group 3 (F+HCTZ): 60% fructose diet plus hydrochlorothiazide (HCTZ, Sigma-Aldrich, St. Louis, Mo.) 10 mg/kg/day dissolved in drinking water

Group 4 (F+HCTZ+KCL): 60% fructose diet plus the same dose of HCTZ and 1% potassium chloride (KCL) dissolved in drinking water. We monitored serum K⁺ and increased the concentration of KCL at week 5 (1.5% KCL), week 15 (1.75% KCL) and week 17 (2% KCL) to maintain normal k⁺ level in serum through the whole study.

Group 5 (F+HCTZ+Allopurinol): 60% fructose diet plus the same dose of HCTZ and allopurinol 150 mg/L dissolved in drinking water

Male Since hypokalemia, which may result from HCTZ use, can reduce the amount of food intake, we pair-fed rats to assure equivalent caloric intake to exclude the influence of different food intake on the metabolic abnormalities.

Rats were weighed weekly and housed separately in metabolic cages for overnight collection of urine (over 18 hrs) with free access to water and no food on weeks 4, 14 and 20. Four hour fasting serum was also collected from the tail vein within the same week of urine collection. At the end of week 20, rats were sacrificed.

Tail Cuff Blood Pressure Measurement

Systolic blood pressure (SBP) measurement was performed in conscious rats with the use of a tail-cuff sphygmomanometer (Visitech BP2000, Visitech Systems, Apex, N.C.) at weeks 4 and 16. After three time periods of preconditioning, rats were placed in prewarmed chambers (37° C.) for 10-15 min, the pressure and pulse were measured by an automatic tail-cuff inflater and a built-in transducer. The mean of three stable BP readings was used.

Biochemical Measurements

Serum and urine K⁺ concentrations were determined with the atomic absorption spectrophotometer (Perkin-Elmer 306). Routine chemistries (including serum glucose, cholesterol, triglycerides, blood urea nitrogen (BUN), sodium, chloride, bicarbonate (HCO₃⁻) and magnesium (Mg⁺⁺); serum and urine creatinine (Cr), uric acid and urine protein concentration) were measured using an autoanalyzer (VetAce, Alfa Wassermann, Inc., West Caldwell, N.J.).

Serum Insulin and the Quantitative Insulin Sensitivity Check Index (QUICKI)

Fasting serum glucose and insulin were obtained at week 14 and used to calculate the QUICKI value. Serum insulin was determined by using the rat insulin ELISA kit (Crystal Chem, Chicago, Ill.) which can detect serum insulin in the range of 156-10,000 pg/ml and has the 3.5% intra-assay and 6.3% inter-assay precision. QUICKI is a mathematical model based on log-transformed fasting plasma glucose and insulin values by equal 1/(log [glucose]+log [insulin]). QUICKI predicts insulin sensitivity with lower values representing more insulin resistance. QUICKI shows good correlation with hyperinsulinemic-euglycemic clamp method, especially in subjects with impaired glucose tolerance (25).

Insulin Tolerance Test (ITT)

At week 18, the ITT was performed on animals fasted for 16 hours by administering 0.75 U/kg recombinant human insulin (Novolin; Novo Nordisk, Princeton, N.J.) via intraperitoneal injection. Blood glucose was determined on tail blood via hand-held blood glucose monitor (OneTouch, Johnson & Johnson) at 5 points; before insulin injection, and at 15, 30, 45 and 60 minutes after insulin injection.

Measurement of Urinary Nitric Oxide

Urine was determined NO by using the nitrate/nitrite colorimetric assay kit (Cayman Chemical Company, MI). The kit measures NO reaction products, total nitrate and nitrite concentrations with a modified two-step Griess reaction.

Statistical Analysis

All data are shown as mean±SD. One way ANOVA (SPSSS 14.0 for window) and post hoc multiple comparisons were used to determine the significance between the mean of multiple groups with the least-significant difference (LSD) test for equal and Dunnett's test for unequal variances. The homogeneity of variance was clarified by Levene's test. The paired and unpaired Student's t test was used to compare the continuous variables of the specific two groups. Pearson Correlation was used to address potential associations between groups. Statistical significance was defined as p<0.05.

Results

Body Weights

Because rats were pair fed and had identical calorie and protein intake, all groups had similar average body weights at week 20 (N; 543±45, F; 565±66, F+HCTZ; 560±88, F+HCTZ+KCL; 558±58 and F+HCTZ+Allopurinol; 557±89 g).

Rats on Fructose Diet Developed the Features of Metabolic Syndrome

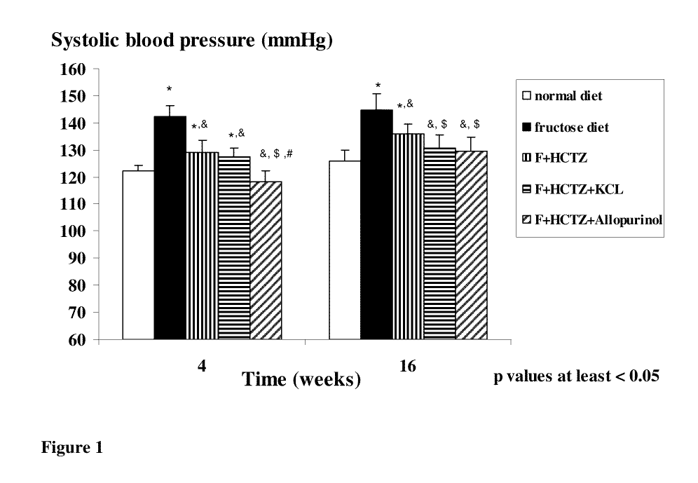

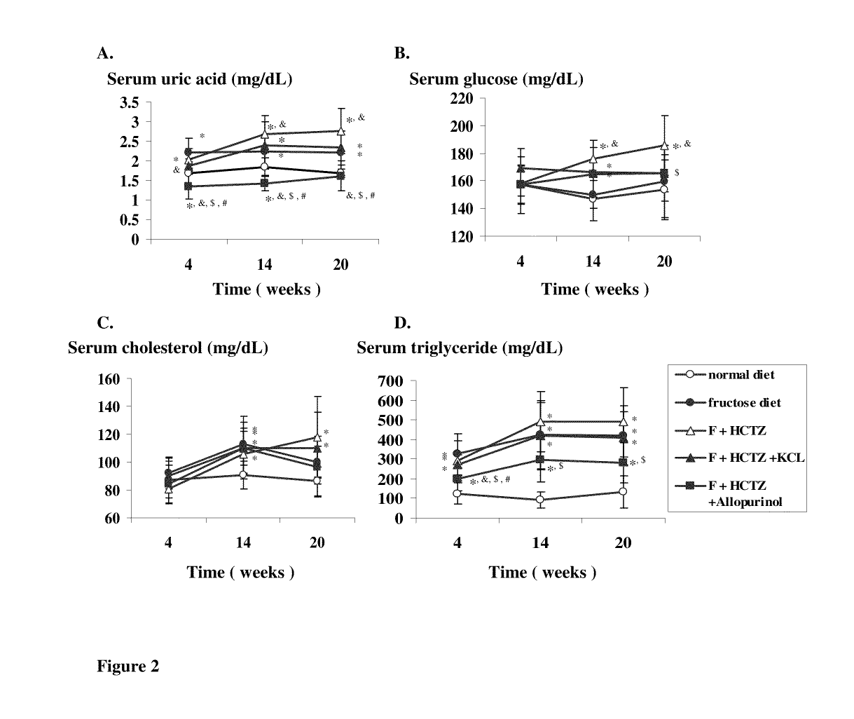

At week 4, the fructose-fed rats (F group) had developed early features of metabolic syndrome including hypertension (SBP: N; 122±1.9 vs. F; 142±4.2 mmHg, p<0.001, FIG. 1), hypertriglyceridemia (N; 125±55 vs. F; 325±104 mg/dL, p<0.001) and hyperuricemia (N; 1.68±0.31 vs. F; 2.2±0.38 mg/dL, p=0.01). Serum cholesterol was significant higher at week 14 (N; 91±10 vs. F group; 113±21 mg/dL, p=0.02). The time course of hypertriglyceridemia, hypercholesterolemia and hyperuricemia are presented in FIG. 2.

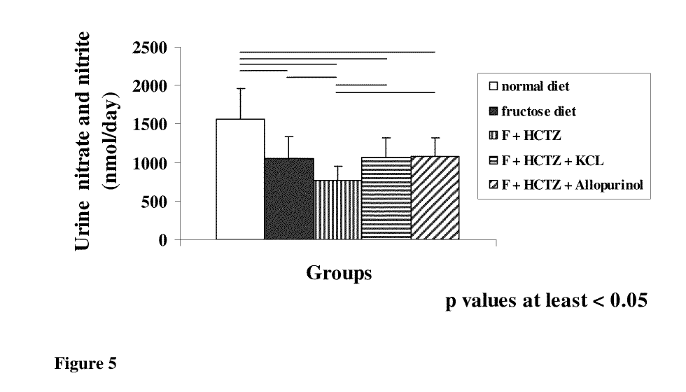

No significant difference in serum glucose was observed between the F group and the N group (FIG. 2). However, there was evidence of insulin resistance as demonstrated by the ITT at week 18 (FIG. 4).

Effect of HCTZ on SBP, serum K⁺ and Mg⁺⁺ with Aggravation of the Metabolic Syndrome

HCTZ usage reduced SBP in the F+HCTZ group compared with the F group (week 4: F; 142±4.2 vs. F+HCTZ; 129±4.6 mmHg, p<0.001 and week 16: F; 145±6.3 vs. F+HCTZ; 136±3.7 mmHg, p=0.01, FIG. 1). Serum K⁺ was significantly lower in the F+HCTZ and F+HCTZ+Allopurinol rats (Table 1). The HCTZ-treated rats (all group 3, 4 and 5) also developed hypomagnesemia with an increase of serum HCO₃⁻ (F; 23.0±0.9, F+HCTZ; 24.7±1.1, F+HCTZ+KCL; 25.6±1.8, F+HCTZ+Allopurinol; 25.7±1.5 mEq/L, all p value <0.05 vs. the F group.)

HCTZ use did not exacerbate metabolic syndrome early (week 4) as the metabolic profile of F group and F+HCTZ group was similar at this time. However, HCTZ aggravated the metabolic syndrome at weeks 14 and 20 with evidence of worse insulin resistance (represented by a significantly lower QUCKI value at week 14, p=0.033, FIG. 2), higher serum uric acid values (F; 2.24±0.45 vs. F+HCTZ; 2.69±0.47 mg/dL, p=0.04 and week 20: F; 2.2±0.56 vs. F+HCTZ; 2.76±0.59 mg/dL, p=0.02) and serum glucose (week 14: F; 150±10.3 vs. F+HCTZ; 176±13.3 mg/dL, p=0.006 and week 20: F; 160±26.4 vs. F+HCTZ; 186±21.9 mg/dL, p=0.02). Serum triglycerides were also higher in the F+HCTZ group but did not reach statistical significance (week 14: F; 424±175 vs. F+HCTZ; 492±154 mg/dL and week 20: F; 419±153 vs. F+HCTZ; 489±178 mg/dL).

Potassium Supplementation Reduced Blood Pressure and Improved Insulin Resistance

At week 4, F+HCTZ+KCL rats had similar SBP compared with F+HCTZ rats (F+HCTZ; 129±4.6 vs. F+HCTZ+KCL 127±3.3 mmHg, p>0.05). However, the effect of potassium supplementation on blood pressure reduction was observed at week 16 (F+HCTZ; 136±3.7 vs. F+HCTZ+KCL 131±4.8 mmHg, p=0.04).

For the ITT performed at week 18, the F+HCTZ+KCL group had a decrease in serum glucose in response to insulin injection while the F+HCTZ group maintained elevated serum glucose in response to insulin, consistent with an effect of potassium supplementation to improve insulin resistance. No significant change of serum uric acid and serum triglycerides in the F+HCTZ+KCL group was observed compared with the F+HCTZ group while there was a tendency of lower serum glucose at week 20 (F+HCTZ; 186±22 vs. F+HCTZ+KCL 166±20 mmHg, p=0.06).

Allopurinol Treatment Improves Hypertension, Hypertriglyceridemia, Hyperglycemia and Insulin Resistance

Serum uric acid of the F+HCTZ+Allopurinol group was equal or lower than N group throughout the study. The SBP of the rats receiving allopurinol at weeks 4 and 16 were lower than the F+HCTZ group (week 4: F+HCTZ; 129±4.6 vs. F+HCTZ+Allopurinol 118±4.2 mmHg, p<0.001 and week 16: F+HCTZ; 136±3.7 vs. F+HCTZ+KCL 130±5.0 mmHg, p=0.02, FIG. 1). Serum triglycerides at weeks 4, 14 and 20 were also significantly lower than the F+HCTZ group (FIG. 2).

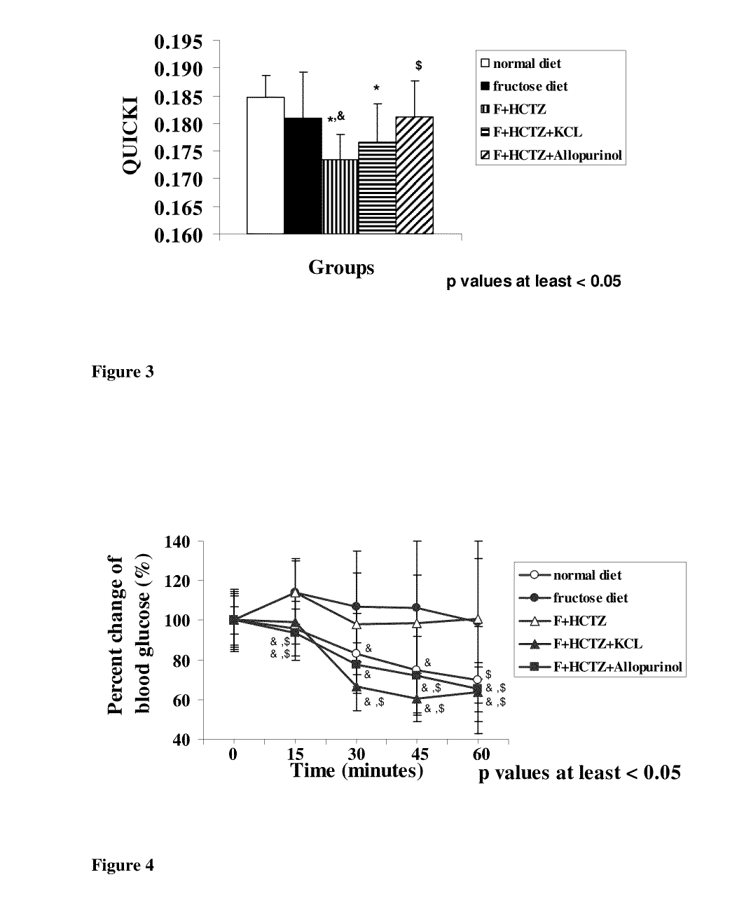

Allopurinol improved insulin resistance evidenced by the higher QUICKI value at week 14 and the higher insulin sensitive response in the ITT compared with the F+HCTZ group (FIGS. 3 and 4). In addition, serum glucose was also lower at week 20 (F+HCTZ; 186±22 vs. F+HCTZ+Allopurinol 166±14 mg/dL, p=0.04).

Serum Insulin Concentrations and Correlation Between Serum Insulin and Serum Glucose, Triglyceride and Cholesterol

Serum insulin concentrations measured at week 14 of the N, F, F+HCTZ, F+HCTZ+KCL and F+HCTZ+Allopurinol groups were 1846±452, 2561±1286, 3449±1200, 3058±1514 and 2319±971 pg/mL, respectively. Significant differences of serum insulin were detected between the F+HCTZ vs. N groups (p=0.002) and borderline significance between the F+HCTZ vs F+HCTZ+Allopurinol groups (p=0.05).

Serum insulin correlated with serum glucose (r=0.65, p<0.001) consistent with the expected relationship of these two parameters. Serum insulin also significantly correlated with serum triglyceride (r=0.59, p<0.001) and serum cholesterol (r=0.35, p=0.033) which also supports an association between insulin resistance and dyslipidemia (26,27).

Correlation Between Serum Uric Acid and Serum Triglyceride, Cholesterol and Glucose, Systolic Blood Pressure and Serum Insulin

When individual rats data at week 20 were examined, serum uric acid positively correlated with serum triglyceride (r=0.77, p<0.001), serum cholesterol (r=0.49, p=0.001) and serum glucose (r=0.46, p=0.003). Furthermore, there was a significant correlation between serum uric acid and SBP at week 4 (r=0.53, p=0.003) and serum insulin at week 14 (r=0.42, p=0.009).

Renal function and urine uric acid excretion

BUN of all HCTZ-treated groups (groups 3, 4 and 5) were significantly higher than N and F groups at all the time points of blood collection, suggesting volume depletion induced by HCTZ. No significant difference of serum Cr between groups was observed at week 20. Increased proteinuria was also observed in all 4 groups compared with N group as shown in the Table 1. The F group had hyperuricemia with higher urine uric acid excreted per day and uric acid clearance compared with the N group which suggested an increase of uric acid production. The F+HCTZ group had greater hyperuricemia with similar reductions of uric acid excretion and uric acid clearance compared with the F group. Treatment with allopurinol had no effect on total urine uric acid excretion per day but did increase uric acid clearance as compared with the F+HCTZ group (Table 1).

Urine NO was Increased with Potassium or Allopurinol Supplement

Because endothelial dysfunction leads to decreased bioavailability of NO (28) and urine nitrate/nitrite excretion is a marker of NO bioavailability (29), we measured urinary nitrate/nitrite as an indirect evidence of endothelial function. Urine nitrate/nitrite was decreased in the F group and the F+HCTZ groups had even lower nitrite excretion. Supplementation of F+HCTZ with potassium or allopurinol increased urine nitrate/nitrite excretion (FIG. 5).

The disclosures of all cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims

### REFERENCES

1. Hall W D, Watkins L O, Wright J T Jr, Wenger N K, Kumanyika S K, Gavin J R 3rd, Ferdinand K C, Watson K, Clark L T, Flack J M, Reed J W, Horton E W, Saunders E; African-American Lipid and Cardiovascular Council. The metabolic syndrome: recognition and management. Dis Manag. 2006; 9:16-33.
2. Peralta C A, Kurella M, Lo J C, Chertow G M. The metabolic syndrome and chronic kidney disease. Curr Opin Nephrol Hypertens. 2006; 15:361-365.
3. Liberopoulos E N, Mikhailidis D P, Elisaf M S. Diagnosis and management of the metabolic syndrome in obesity. Obes Rev. 2005; 6:283-296.
4. Wubben D P, Adams A K. Metabolic syndrome: what's in a name? WMJ. 2006; 105:17-20.
5. Nakagawa T, Hu H, Zharikov S, Tuttle K R, Short R A, Glushakova O, Ouyang X, Feig D I, Block E R, Herrera-Acosta J, Patel J M, Johnson R J. A causal role for uric acid in fructose-induced metabolic syndrome. Am J Physiol Renal Physio.l 2006; 290:F625-F631.
6. Deedwania P C. Mechanisms of endothelial dysfunction in the metabolic syndrome. Curr Diab Rep. 2003; 3:289-292.
7. Expert Panel on Detection, Evaluation, and Treatment of High Blood Cholesterol in Adults. Executive Summary of The Third Report of The National Cholesterol Education Program (NCEP) Expert Panel on Detection, Evaluation, And Treatment of High Blood Cholesterol In Adults (Adult Treatment Panel III). JAMA. 2001; 285:2486-2497.
8. Grundy S M, Cleeman J I, Daniels S R, Donato K A, Eckel R H, Franklin B A, Gordon D J, Krauss R M, Savage P J, Smith S C Jr, Spertus J A, Costa F; American Heart Association; National Heart, Lung, and Blood Institute. Diagnosis and management of the metabolic syndrome: an American Heart Association/National Heart, Lung, and Blood Institute Scientific Statement. Circulation. 2005; 112:2735-2752.
9. Grundy S M. Does the metabolic syndrome exist? Diabetes Care. 2006; 29:1689-1692.
10. Collins R, Peto R, MacMahon S, Hebert P, Fiebach N H, Eberlein K A, Godwin J, Qizilbash N, Taylor J O, Hennekens C H. Blood pressure, stroke, and coronary heart disease. Part 2, short-term reductions in blood pressure: overview of randomized drug trials in their epidemiological context. Lancet. 1990; 335:827-838.
11. Turnbull F. Effects of different blood-pressure-lowering regimens on major cardiovascular events: results of prospectively-designed overviews of randomized trials. Lancet. 2003; 362:1527-1535.
12. The ALLHAT Study Group. Major outcomes in high-risk hypertensive patients randomized to angiotensin-converting enzyme inhibitor or calcium-channel blocker vs. diuretic. JAMA. 2002; 288:2981-2997.
13. Brown M J, Palmer C R, Castaigne A, de Leeuw P W, Mancia G, Rosenthal T, Ruilope L M. Morbidity and mortality in patients randomized to double-blind treatment with a long acting calcium-channel blocker or diuretic in the international nifedipine GITS study: Intervention as a Goal in Hypertension Treatment (INSIGHT). Lancet. 2000; 356:366-372.
14. Plavinik F L, Rodrigues C I, Zanella M T, Ribeiro A B. Hypokalemia, glucose intolerance, and hyperinsulinemia during diuretic therapy. Hypertension. 1992; 19 suppl 2:1126-1129.
15. Punzi H A, Punzi C F; Antihypertensive and Lipid-Lowering Heart Attack Trial Study; Trinity Hypertension Research Institute. Metabolic issues in the Antihypertensive and Lipid-Lowering Heart Attack Trial Study. Curr Hypertens Rep. 2004; 6:106-110.
16. Verdecchia P, Reboldi G, Angeli F, Borgioni C, Gattobigio R, Filippucci L, Norgiolini S, Bracco C, Porcellati C. Adverse prognostic significance of new diabetes in treated hypertensive subjects. Hypertension. 2004; 43:963-969.
17. Franse L V, Pahor M, Di Bari M, Shorr R I, Wan J Y, Somes G W, Applegate W B. Serum uric acid, diuretic treatment and risk of cardiovascular events in the Systolic Hypertension in the Elderly Program. J Hypertens. 2000; 18:1149-54.
18. Amery A, Birkenhager W, Brixko P, Bulpitt C, Clement D, Deruyttere M, De Schaepdryver A, Fagard R, Forette F, Forte J, et al. Glucose intolerance during diuretic therapy in elderly hypertensive patients. A second report from the European Working Party on high blood pressure in the elderly (EWPHE). Postgrad Med J. 1986; 62:919-924.
19. Andersson O K, Gudbrandsson T, Jamerson K. Metabolic adverse effects of thiazide diuretics: the importance of normokalaemia. J Intern Med Suppl. 1991; 735:89-96.
20. Khosla U M, Zharikov S, Finch J L, Nakagawa T, Roncal C, Mu W, Krotova K, Block E R, Prabhakar S, Johnson R J. Hyperuricemia induces endothelial dysfunction. Kidney Int. 2005; 67:1739-1742.
21. Mazzali M, Hughes J, Kim Y G, Jefferson J A, Kang D H, Gordon K L, Lan H Y, Kivlighn S, Johnson R J. Elevated uric acid increases blood pressure in the rat by a novel crystal-independent mechanism. Hypertension. 2001; 38:1101-1106.
22. Nakagawa T, Tuttle K R, Short R A, Johnson R J. Hypothesis: fructose-induced hyperuricemia as a causal mechanism for the epidemic of the metabolic syndrome. Nat Clin Pract Nephrol. 2005; 1:80-86.

TABLE 1
Renal function, serum K⁺, serum Mg⁺⁺ and urinary uric acid excretion

| | | | | F + HCTZ + | F + HCTZ + |
|---|---|---|---|---|---|
| Groups | N | F | F + HCTZ | KCL | Allopurinol |
| BUN | 13.8 ± 1.4 | 13 ± 2.6 | 26.6 ± 14 *^{, &} | 25.3 ± 6.6 *^{, &} | 22.5 ± 6.4 *^{, &} |
| (mg/dL) | | | | | |
| Cr (mg/dL) | 0.44 ± 0.05 | 0.43 ± 0.05 | 0.46 ± 0.11 | 0.44 ± 0.07 | 0.44 ± 0.05 |
| Serum K⁺ | 4.4 ± 0.2 | 4.3 ± 0.1 | 4.0 ± 0.1 *^{, &, #} | 4.5 ± 0.3 | 4.1 ± 0.1 *^{, &, #} |
| (mEq/L) | | | | | |
| Serum | 1.6 ± 0.28 | 1.5 ± 0.23 | 1.15 ± 0.1*^{, &} | 1.09 ± 0.1 *^{, &} | 1.1 ± 0.1 *^{, &} |
| Mg⁺⁺ | | | | | |
| (mEq/L) | | | | | |
| Urine | 0.24 ± 0.4 | 2.29 ± 2.5 * | 2.53 ± 2.3 * | 3.18 ± 2.8 * | 2.86 ± 2.4 * |
| protein/Cr | | | | | |
| Urine uric | 2.42 ± 0.4 | 4.07 ± 0.8 * | 2.93 ± 1.0 ^{&} | 3.67 ± 0.9 * | 3.38 ± 1.1 * |
| acid | | | | | |
| (mg/day) | | | | | |
| Uric acid | 0.09 ± 0.02 | 0.13 ± 0.02 * | 0.08 ± 0.03 ^{&} | 0.11 ± 0.3 | 0.17 ± 0.05 *^{, $, #} |
| clearance | | | | | |
| (ml/min) | | | | | |

Data are expressed as means ± SD. N, normal diet group; F, fructose diet group; F + HCTZ, fructose diet and hydrochlorothiazide group; F + HCTZ + KCL, fructose diet and hydrochlorothiazide plus potassium chloride group; F + HCTZ + Allopurinol, fructose diet and hydrochlorothiazide plus allopurinol group.
p values at least <0.05; * p vs normal diet, ^{&} p vs fructose diet, ^{$} p vs F + HCTZ, ^{#} p vs F + HCTZ + KCL

What is claimed is:

1. A reduced-lipid producing thiazide containing composition consisting essentially of a thiazide or pharmaceutically acceptable salt thereof, allopurinol, or a pharmaceutically acceptable salt thereof, and a non-active additive.

2. The composition of claim 1, wherein said thiazide is chlorothiazide, benzylhydro-chlorothiazide, cyclopenthiazide, ethiazide, hydrochlorothiazide, hydroflumethiazide, methyclothiazide, penfluthiazide, polythiazide, or trichloromethiazide or a combination thereof.

3. The composition of claim 1, wherein said thiazide is hydrochlorothiazide.

4. The composition of claim 1 formulated in solid dosage form comprising a powder, granule, tablet, pill, or capsule.

5. The composition of claim 1 formulated in a liquid suspension or solution.

6. A method of treating hypertension comprising administering a therapeutically effective amount of a thiazide, or pharmaceutically acceptable salt thereof, and coadministering a therapeutically effective amount of allopurinol, or pharmaceutically acceptable salt thereof, wherein coadministering comprises administering an amount of allopurinol sufficient to reduce lipid-producing effects of thiazide.

7. The method of claim 6, wherein said allopurinol is administered simultaneously with said thiazide.

8. The method of claim 6, wherein said allopurinol is administered before said thiazide.

9. The method of claim 6, wherein said allopurinol is administered after said thiazide.

10. The method of claim 6, wherein said thiazide is chlorothiazide, benzylhydro-chlorothiazide, cyclopenthiazide, ethiazide, hydrochlorothiazide, hydroflumethiazide, methyclothiazide, penfluthiazide, polythiazide, or trichloromethiazide or a combination thereof.

11. The method of claim 6, wherein said thiazide is hydrochlorothiazide, or a pharmaceutically acceptable salt thereof.

12. The method of claim 6, wherein said thiazide, or pharmaceutically acceptable salt thereof and said allopurinol, or pharmaceutically acceptable salt thereof, are administered orally.

13. The method of claim 6, wherein said thiazide, or pharmaceutically acceptable salt thereof, or allopurinol, or pharmaceutically acceptable salt thereof, are administered orally.

14. The method of claim 6, wherein said thiazide, or pharmaceutically acceptable salt thereof, or allopurinol, or pharmaceutically acceptable salt thereof, are administered parenterally.

15. The method of claim 6, wherein said thiazide, or pharmaceutically acceptable salt thereof, and allopurinol, or pharmaceutically acceptable salt thereof, are administered together in a solid dosage form comprising a powder, granule, tablet, pill, or capsule.

16. A method of treating or preventing hypertension in a patient in need thereof comprising administering a therapeutically effective amount of a composition of claim 1.

17. A method of reducing thiazide induced hyperlipidemia in a patient in which a thiazide is being administered comprising coadministering a therapeutically effective amount of allopurinol, or pharmaceutically acceptable salt thereof, wherein said therapeutically effective amount is sufficient to reduce the lipid producing effect of said thiazide.
